(19) United States
(12) Patent Application Publication
(10) Pub. No.: US 2026/0174944 A1
(43) Pub. Date: Jun. 25, 2026

(54) REUSE PREVENTION FOR DIALYSIS BAGS

(71) Applicant: Fresenius Medical Care Holdings, Inc., Waltham, MA (US)

(72) Inventors: David Yuds, Hudson, NH (US); Diego Suarez del Real Pena, Mission, TX (US); Jose Omar Aguilar Flores, Mission, TX (US)

(21) Appl. No.: 19/414,924

(22) Filed: Dec. 10, 2025

Related U.S. Application Data
(60) Provisional application No. 63/736,170, filed on Dec. 19, 2024.

(51) Int. Cl. A61M 1/28; A61M 1/00; A61M 39/00; A61M 39/10

(52) U.S. Cl. CPC A61M 1/28; A61M 1/69; A61M 39/1011; A61M 2039/0009; A61M 2039/1005; A61M 2039/1027; A61M 2039/1061; A61M 2202/0007; A61M 2202/0014; A61M 2202/04; A61M 2205/273; A61M 2205/584; A61M 2210/1017

(57) ABSTRACT

The present teachings generally include devices, systems, methods, and kits featuring safeguards to prevent the accidental reuse of effluent-filled bags as a source of fresh dialysis solution in peritoneal dialysis systems. This may include, for example, bags with chemical sensors that react with effluent contents to trigger visual indicators that alert a user to the bag being unsuitable for (re) use as dialysis solution. The present teachings may also or instead include bags engaged with connectors that mechanically alter and/or permanently couple during a dialysis treatment to prevent recoupling of the bags to supply tubing in the system, and/or techniques to ensure proper use of dialysis bags throughout a treatment cycle. These features can be useful for enhancing patient safety, particularly for users with cognitive impairments or in settings with limited oversight, e.g., by providing clear visual cues and/or physical barriers preventing potentially harmful misuse of dialysis bags.

### CROSS-REFERENCE TO RELATED APPLICATIONS

This application claims priority to U.S. Provisional Pat. App. No. 63/736,170 filed on Dec. 19, 2024, the entire contents of which are hereby incorporated by reference.

### FIELD

The present disclosure generally relates to devices, systems, kits, and methods featuring dialysis bags—e.g., for peritoneal dialysis systems-that include one or more features to prevent the use of effluent-filled bags as a dialysate supply.

### BACKGROUND

Peritoneal dialysis is a widely used treatment for patients with kidney failure, allowing them to perform dialysis at home rather than visiting a clinic multiple times per week. The process involves cycling dialysis fluid in and out of the patient's peritoneal cavity to remove waste products from their blood. While peritoneal dialysis offers greater flexibility and independence for patients, it requires careful management of equipment and supplies to ensure safe and effective treatment.

Some available peritoneal dialysis systems allow patients to reuse dialysis solution bags, once emptied, as drain bags to drain effluent from the patient's peritoneum. One such system is the SILENCIA system from Fresenius Medical Care (see, e.g., U.S. Pat. No. 11,612,680, which is hereby incorporated by reference herein). By way of example, in such a system, fresh dialysis solution bags are placed in a tray on the top of a machine, while drain bags for collecting effluent drained from the patient are placed in a lower tray of the machine. The two sets of bags are connected by an integrated “Vario-Connector” and tubing system with a Y-connector that leads to the patient with automatic mechanical clamps on either end. In particular, the Vario connector allows a user to run a treatment and then, when the fresh dialysate bags are empty at the end of the treatment, break off the tubing with the empty dialysate bags to be used as the drain bags for the next treatment. However, a challenge in such a system, and in other similar peritoneal dialysis systems, is preventing the accidental reuse of bags that have been filled with effluent as a source of fresh dialysis solution. Such misuse could lead to serious complications for a patient. Current systems rely heavily on proper training and vigilance from users to avoid this error. However, as peritoneal dialysis becomes more widely adopted, including use by patients with cognitive impairments or in settings with less oversight, there is a growing need for foolproof safeguards against the improper reuse of dialysis bags.

There remains a need for improved safeguards in peritoneal dialysis systems and similar, e.g., to prevent the accidental reuse of effluent-filled bags as a source of fresh dialysis solution.

### SUMMARY

The present teachings generally include devices, systems, methods, and kits featuring safeguards to prevent the accidental reuse of effluent-filled bags as a source of fresh dialysis solution in peritoneal dialysis systems. This may include, for example, bags with chemical sensors that react with effluent contents to trigger visual indicators that alert a user to the bag being unsuitable for (re) use as dialysis solution. The present teachings may also or instead include bags engaged with connectors that mechanically alter and/or permanently couple during a dialysis treatment to prevent recoupling of the bags to supply tubing in the system, and/or techniques to ensure proper use of dialysis bags throughout a treatment cycle. These features can be useful for enhancing patient safety, particularly for users with cognitive impairments or in settings with limited oversight, e.g., by providing clear visual cues and/or physical barriers preventing potentially harmful misuse of dialysis bags.

In an example aspect, a peritoneal dialysis system may include: supply side tubing with a supply side connector structurally configured to fluidically connect to a supply of dialysis solution to transfer the dialysis solution to a peritoneum of a patient; drain side tubing with a drain side connector structurally configured to fluidically connect to a drainage receptacle to transfer effluent from the peritoneum of the patient to the drainage receptacle; and a bag including a first connector structurally configured such that the bag is couplable only once to the supply side connector such that the bag, when filled with fresh dialysis solution, is configured to be the supply of dialysis solution in the system, the bag further structurally configured for fluidically coupling to the drain side connector after being coupled to the supply side connector such that the bag, when emptied, is configured to be the drainage receptacle for effluent but thereafter cannot subsequently be reconnected to the supply side tubing.

Implementations of this example aspect, or any other example aspect described in this summary section or otherwise herein, may include one or more of the following features. The first connector may be mechanically altered upon engagement with the supply side connector such that, upon disengagement from the supply side connector, the first connector cannot be reengaged with the supply side connector. The first connector may include a first feature and the supply side connector may include a second feature, the second feature structurally configured to engage with the first feature and to move the first feature from an initial position to a final position through one or more of an engagement operation and a disengagement operation, where the final position of the first feature prevents reengagement between the first connector and the supply side connector. The first feature may include a ring with a notch, and the second feature may include a tab, where the tab is engageable with the notch when the first connector and the supply side connector are fully seated relative to one another, and where, when the supply side connector is disengaged from the first connector, the tab moves the ring outward towards an opening in the first connector thereby at least partially blocking the opening from receiving the supply side connector. The first feature may be configured to move along a length of the first connector in a direction substantially parallel to a central axis of the first connector from a first axial position to a second axial position, the first axial position more proximal to a base portion of the first connector, and more distal to an opening of the first connector, than the second axial position. The second feature may engage with a notch of the first feature when the first feature is in the first axial position, where interaction between the second feature and the notch during disengagement causes the first feature to translate from the first axial position to the second axial position. The first feature may be prevented from moving back to the first axial position once positioned in the second axial position. The first feature may include one or more of a ring-like structure, a collar, a sleeve, an annular member, a segmented ring structure, a C-shaped element, and multiple discrete elements arranged circumferentially within the first connector. The first connector, when engaged with the supply side connector, may become permanently coupled to the supply side connector, and the system further may include a downstream connector that is couplable to the drain side connector for use of the bag, when emptied, as the drainage receptacle. The first connector may include a first feature and the supply side connector may include a second feature, the first feature and the second feature configured to engage such that, when the first connector and the supply side connector are fully seated relative to one another, the first feature and the second feature permanently lock engagement therebetween. The first feature may include a notch and the second feature may include a catch. The catch may be included on an angled barb. One or more of the supply side connector and the first connector may include a breakpoint disposed away from one or more coupled portions of the first connector and the supply side connector. The breakpoint may be thinner than at least one of the one or more coupled portions of the first connector and the supply side connector. The system may include a chemical sensor in an interior of the bag, the chemical sensor reactive with one or more contents of the effluent but non-reactive with the fresh dialysis solution. The chemical sensor may include, or may be in communication with, a visual indicator that changes from a first visual state to a second visual state when the chemical sensor reacts with the one or more contents of the effluent to visually indicate that the bag contains waste.

In an example aspect, a method of preventing reuse of an effluent bag as a dialysate supply may include: providing a bag structurally configured to connect with each of supply side tubing and drain side tubing in a peritoneal dialysis system, the bag including fresh dialysis solution; fluidically connecting the bag with the supply side tubing and supplying the fresh dialysis solution from the bag to a peritoneum of a patient during a first sequence of operation in a dialysis procedure, thereby substantially emptying the bag; fluidically connecting the bag with the drain side tubing and receiving effluent from the peritoneum of the patient during a second sequence of operation in the dialysis procedure, thereby substantially filling the bag with effluent; disconnecting the bag from the drain side tubing; and preventing reconnection of the bag to the supply side tubing.

Implementations of this example aspect, or any other example aspect described in this summary section or otherwise herein, may include one or more of the following features. Fluidically connecting the bag with the supply side tubing may include mechanically altering a first connector of the bag with a supply side connector such that, when the first connector is disengaged from the supply side connector, the first connector cannot be reengaged with the supply side connector. Fluidically connecting the bag with the supply side tubing may include permanently coupling a first connector of the bag with a supply side connector, the peritoneal dialysis system further including a tubing connector disposed downstream from the supply side connector. Fluidically connecting the bag with the drain side tubing may include disconnecting the tubing connector from supply side tubing and connecting the tubing connector to the drain side tubing, where disconnecting the bag from the drain side tubing includes decoupling the tubing connector from drain side tubing. The method may include breaking at least a portion of the connection between the first connector and the supply side connector to separate the bag from tubing. The method may include disposing of effluent in the bag.

In an example aspect, a bag for a peritoneal dialysis system may include: an exterior surface; a void in an interior of the bag structurally configured to be filled with a fluid; and a first connector in fluid communication with the void, the first connector structurally configured such that the bag is couplable only once to a supply side connector to transfer a supply of dialysis solution from the void to a peritoneum of a patient, where the bag is structurally configured for fluidically coupling to a drain side connector after being coupled to the supply side connector such that the bag, when emptied, is a drainage receptacle for effluent but thereafter cannot subsequently be reconnected to supply side tubing.

In an example aspect, a peritoneal dialysis system may include: supply side tubing structurally configured to fluidically connect a supply of dialysis solution to a peritoneum of a patient; drain side tubing structurally configured to fluidically connect the peritoneum of the patient to a drainage receptacle; and a bag engaged with one or more connectors structurally configured to independently connect with each of the supply side tubing and the drain side tubing such that the bag, when filled with fresh dialysis solution and connected with the supply side tubing, is the supply of dialysis solution in the system, and the bag, when emptied and connected with the drain side tubing, is the drainage receptacle to receive effluent in the system. The bag may include a chemical sensor in an interior thereof, the chemical sensor reactive with one or more contents of the effluent but non-reactive with the fresh dialysis solution, the chemical sensor including, or in communication with, a visual indicator that changes from a first visual state to a second visual state when the chemical sensor reacts with the one or more contents of the effluent to visually indicate that the bag contains waste for preventing subsequent use of the bag as the supply of dialysis solution in the system.

Implementations of this example aspect, or any other example aspect described in this summary section or otherwise herein, may include one or more of the following features. The first visual state may include an absence of visual indication, and the second visual state may include a presence of a visual indication. The visual indication may include one or more of a color, a symbol, text, patterning, and an image. The second visual state may include a color change from the first visual state. The color change may affect a portion of a surface of the bag. The color change may form one or more of a symbol and text visible on the surface of the bag. The color change may affect appearance of contents of the bag. Contents of the bag may appear more opaque in response to the color change. The one or more contents of the effluent may include at least one of urea and creatinine. The one or more contents of the effluent may include a threshold amount of electrolytes. The one or more contents of the effluent may include at least one of a metabolic acid product, aluminum, copper, zinc, a medication, and an inflammatory marker. The chemical sensor may include litmus. The chemical sensor may include one or more of a biosensor, an electrochemical sensor, and a potentiometric sensor. The one or more connectors of the bag may include a first connector that is mechanically altered upon engagement with a supply side connector such that, upon disengagement from the supply side connector, the first connector cannot be reengaged with the supply side connector. The first connector may include a first feature and the supply side connector may include a second feature, the second feature structurally configured to engage with the first feature and to move the first feature from an initial position to a final position through one or more of an engagement operation and a disengagement operation, where the final position of the first feature prevents reengagement between the first connector and the supply side connector. The first connector, when engaged with the supply side connector, may become permanently coupled to the supply side connector, where the system further may include a downstream connector that is couplable to the drain side tubing for use of the bag, when emptied, as the drainage receptacle.

In an example aspect, a peritoneal dialysis system may include: supply side tubing with a supply side connector structurally configured to fluidically connect to a supply of dialysis solution to transfer the dialysis solution to a peritoneum of a patient; drain side tubing with a drain side connector structurally configured to fluidically connect to a drainage receptacle to transfer effluent from the peritoneum of the patient to the drainage receptacle; and a bag. The bag may include: a first connector structurally configured such that the bag is couplable only once to the supply side connector such that the bag, when filled with fresh dialysis solution, is configured to be the supply of dialysis solution in the system, the bag further structurally configured for fluidically coupling to the drain side connector after being coupled to the supply side connector such that the bag, when emptied, is configured to be the drainage receptacle for effluent but thereafter cannot subsequently be reconnected to the supply side tubing; and a chemical sensor in an interior thereof, the chemical sensor reactive with one or more contents of the effluent but non-reactive with the fresh dialysis solution, the chemical sensor including, or in communication with, a visual indicator that changes from a first visual state to a second visual state when the chemical sensor reacts with the one or more contents of the effluent to visually indicate that the bag contains waste.

In an example aspect, a method of identifying a drainage receptacle for dialysis may include providing a bag structurally configured to connect with each of supply side tubing and drain side tubing in a peritoneal dialysis system, the bag including: fresh dialysis solution; a chemical sensor in an interior thereof; and a visual indicator in communication with the chemical sensor, the visual indicator in a first visual state when the bag contains the fresh dialysis solution. The method may also include: fluidically connecting the bag with the supply side tubing and supplying the fresh dialysis solution from the bag to a peritoneum of a patient during a first sequence of operation in a dialysis procedure, thereby substantially emptying the bag; fluidically connecting the bag with the drain side tubing and receiving effluent from the peritoneum of the patient during a second sequence of operation in the dialysis procedure, thereby substantially filling the bag with effluent; detecting, via the chemical sensor, one or more contents of the effluent; and triggering a change in the visual indicator from the first visual state to a second visual state to visually indicate that the bag contains effluent.

In an example aspect, a bag for a peritoneal dialysis system may include: an exterior surface; a void in an interior of the bag structurally configured to be filled with a fluid; one or more connectors in fluid communication with the void, at least one of the one or more connectors structurally configured to independently connect with each of (i) supply side tubing fluidically connecting a supply of dialysis solution to a peritoneum of a patient, and (ii) drain side tubing fluidically connecting the peritoneum of the patient to a drainage receptacle, where the bag, when filled with fresh dialysis solution and connected with the supply side tubing, is configured to be the supply of dialysis solution in a peritoneal dialysis system, and where the bag, when emptied and connected with the drain side tubing, is configured to be the drainage receptacle to receive effluent in the peritoneal dialysis system; a chemical sensor in the interior of the bag, the chemical sensor reactive with one or more contents of the effluent but non-reactive with the fresh dialysis solution; and a visual indicator responsive to the chemical sensor, the visual indicator configured to change from a first visual state to a second visual state when the chemical sensor reacts with the one or more contents of the effluent to visually indicate that the bag contains waste for preventing subsequent use of the bag as the supply of dialysis solution in the peritoneal dialysis system.

These and other features, aspects, and advantages of the present teachings will become better understood with reference to the following description, examples, and appended claims.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1A shows a peritoneal dialysis system, where bags of dialysis solution are engageable to supply side tubing for supplying dialysis solution in the system, according to a representative example.

FIG. 1B shows a peritoneal dialysis system, where bags of dialysis solution are fluidically connected to supply side tubing, according to a representative example.

FIG. 1C shows a peritoneal dialysis system, where bags are engageable to drain side tubing to collect effluent in the system, according to a representative embodiment.

FIG. 1D shows a peritoneal dialysis system that demonstrates how bags used for drainage receptacles should not be reconnected to supply side tubing, according to a representative example.

FIG. 2 shows a bag in two different visual states, according to a representative example.

FIG. 3 is a flow chart of a method of identifying a drainage receptacle for dialysis, according to a representative example.

FIG. 4 shows a typical bag and tubing connector.

FIG. 5 shows a first connector and a supply side connector, according to a representative example.

FIG. 6 shows a first connector engaged with a supply side connector, according to a representative example.

FIG. 7 shows a first connector and a supply side connector, according to a representative example.

FIG. 8 shows a first connector engaged with a supply side connector, according to a representative example.

FIG. 9 shows a first connector and a supply side connector, according to a representative example.

FIG. 10 shows a first connector being prevented from engagement with a supply side connector, according to a representative example.

FIG. 11 shows a first connector and system tubing, according to a representative example.

FIG. 12 shows a first connector engaged with a system connector, according to a representative example.

FIG. 13 is a flow chart of a method of preventing reuse of an effluent bag as a dialysate supply, according to a representative example.

### DETAILED DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately” or the like, when accompanying a numerical value, are to be construed as indicating a deviation as would be appreciated by one of ordinary skill in the art to operate satisfactorily for an intended purpose. Similarly, words of approximation such as “about,” “approximately,” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” and the like, are words of convenience and are not to be construed as limiting terms unless specifically stated to the contrary.

The present teachings generally relate to preventing the accidental reuse of effluent-filled bags as a source of fresh dialysis solution in peritoneal dialysis treatments and similar. More specifically, the present teachings may include peritoneal dialysis bags with sensors (e.g., chemical sensors) that react with effluent contents to trigger alerts (e.g., visual indicators), connectors that mechanically alter and/or permanently couple to prevent reuse of bags as a supply of dialysate under certain conditions, and/or methods for ensuring the proper use of dialysis bags throughout a dialysis treatment cycle. The present teachings may also or instead include procedural safeguards and/or physical barriers to prevent improper reconnection of used bags to the supply side of peritoneal dialysis systems. These features can be particularly useful for enhancing patient safety in home dialysis settings and/or for users with cognitive impairments, by providing clear alerts (e.g., visual cues) and/or mechanical safeguards to protect against the misuse of dialysis bags.

In some aspects, the present teachings may be compatible with existing peritoneal dialysis systems, such as the SILENCIA system from Fresenius Medical Care. For example, bags according to the present teachings may incorporate features that work in conjunction with Fresenius's “Vario-Connectors” or similar integrated tubing systems. In some implementations, the bags may thus include, directly or indirectly, tubing with connectors that are compatible with Vario connectors but that also incorporate, directly or indirectly, the mechanical alteration and/or permanent coupling features described in the present teachings. This compatibility may allow for the adoption of additional safety features in systems already in use, potentially improving patient safety across a wide range of peritoneal dialysis setups. The bags may also be designed to work with automated peritoneal dialysis machines, for example, with visual indicators positioned to be easily readable, e.g., by a user and/or by a machine's optical sensors. The connectors on the bags may be designed with specific keying or coding features that ensure they can only be connected to the appropriate ports on a dialysis machine, further reducing the risk of misuse. Additionally, the bags may be manufactured with materials that are compatible with sterilization methods commonly used in medical settings, e.g., ensuring that the sensors and visual indicators remain functional after sterilization processes.

Therefore, the present disclosure may relate to peritoneal dialysis systems, connectors, bags, and methods for preventing accidental reuse of effluent-filled bags as a source of fresh dialysis solution. However, it will be understood that aspects of the present teachings are not limited to the use case of peritoneal dialysis systems, and that the bags and techniques as disclosed herein may be used in other settings without departing from the scope of this disclosure. Thus, even though the present teachings may emphasize and refer to “peritoneal dialysis” or more generally to “dialysis” related systems and components, other types of systems and components are also or instead possible unless expressly stated to the contrary or otherwise clear from the context.

In some aspects, peritoneal dialysis bags may include sensors (e.g., chemical sensors) in their interior that react with one or more contents of effluent but are non-reactive with fresh dialysis solution. The sensors may be in communication with visual indicators that change from a first visual state to a second visual state when the sensors react with the effluent contents. This visual change may alert users that a bag contains waste, thereby preventing subsequent use of the bag as a supply of dialysis solution. For example, the sensors and visual indicators described herein may be integrated into bags designed for use with such systems, providing an additional layer of safety without requiring significant modifications to existing equipment. In certain aspects, the bags may include RFID tags or other electronic identifiers that can communicate with a dialysis machine, providing information about the bag's contents and usage history. In addition to or instead of a change in a visual state and/or a visual indicator, other alerts and notifications may be used, for example including audio alerts, tactile alerts, communication transmissions, and the like.

Also or instead, in some aspects, peritoneal dialysis systems may include supply side tubing with supply side connectors and drain side tubing with drain side connectors. Bags used in these systems may include first connectors structurally configured to couple only once to the supply side connectors. After being used to supply fresh dialysis solution, the bags may be configured for fluidic coupling to the drain side connectors to serve as drainage receptacles for effluent. The first connectors may be designed such that, after this use cycle, the bags cannot physically be subsequently reconnected to the supply side tubing. By way of example, in certain implementations, the connectors engaged with the bags may be mechanically altered upon engagement with supply side connectors, where this alteration may prevent reengagement between the first connectors and the supply side connectors after initial use. Alternatively, the bag's connectors may become permanently coupled to the supply side connectors when engaged, with downstream connectors provided for subsequent connection to drain side tubing.

Methods for identifying drainage receptacles and preventing the reuse of effluent bags as a dialysate supply are also provided herein. These methods may involve using bags with sensors and visual indicators or other alerts, in addition to or instead of employing mechanical connection mechanisms that prevent the improper reuse of bags. Such approaches may enhance patient safety, particularly in home dialysis settings or for users with cognitive impairments, by providing clear alerts and/or mechanical safeguards against the misuse of dialysis bags.

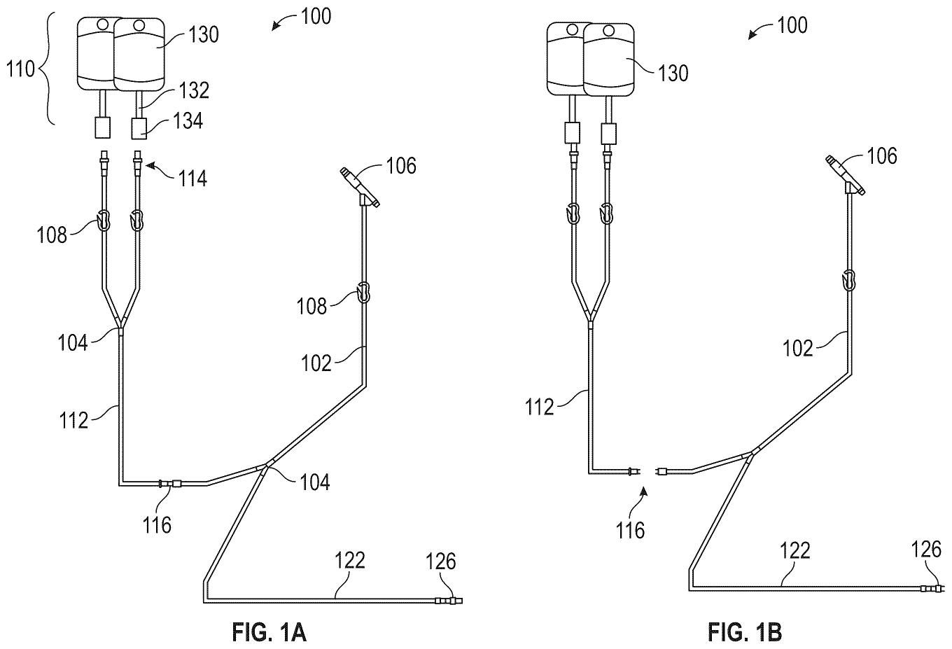

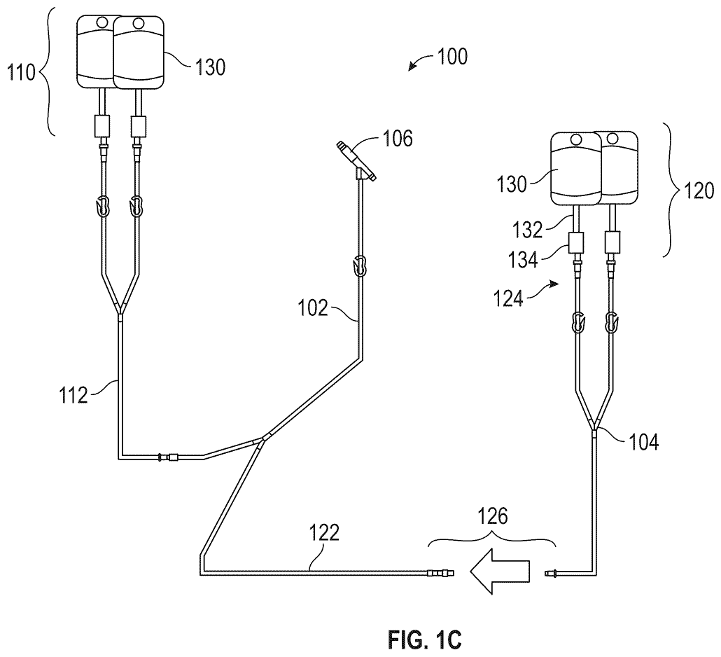

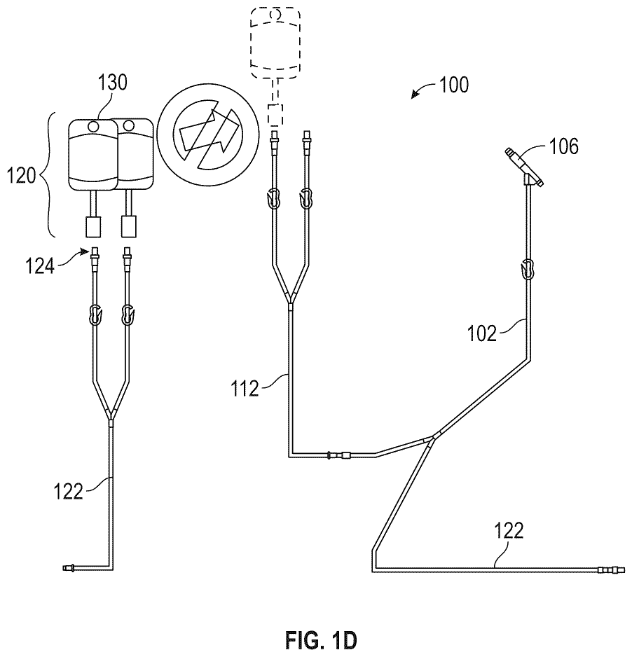

FIGS. 1A-1D generally show a peritoneal dialysis system 100 where bags 130 filled with fresh dialysate can be used first as a supply of dialysis solution 110 in the system 100, and then subsequently, when emptied, the bags 130 can be reused as the drainage receptacle 120 to receive effluent in the system 100. In particular, FIG. 1A shows a peritoneal dialysis system 100, where bags 130 of dialysis solution are engageable to supply side tubing 112 for supplying dialysis solution in the system 100, according to a representative example; FIG. 1B shows a peritoneal dialysis system 100, where bags 130 of dialysis solution are fluidically connected to supply side tubing 112, according to a representative example; FIG. 1C shows a peritoneal dialysis system 100, where bags 130 are engageable to drain side tubing 122 to collect effluent in the system 100, according to a representative embodiment; and FIG. 1D shows a peritoneal dialysis system 100 that demonstrates how bags 130 used for drainage receptacles 120 should not be reconnected to supply side tubing 112, according to a representative example.

Referring to FIGS. 1A and 1B, the system 100 may include several components arranged to facilitate a dialysis treatment including a supply of dialysis solution 110 (i.e., bags 130 filled with fresh dialysate) with bag tubing 132 and bag connectors 134 structurally configured to engage with supply side connectors 114 in line with supply side tubing 112.

As shown in the figure, in some aspects, the system 100 may feature one or more Y-connectors 104. By way of example, a Y-connector 104 may be used to combine tubing from multiple bags 130 into a supply line. Also or instead, the supply side tubing 112 may extend downward and connect to a Y-connector 104 that may serve as a junction point joining the supply side tubing 112 with patient tubing 102 and/or drain side tubing 122. In certain aspects, a downstream connector 116 (e.g., a Vario connector as described above) may be positioned on the supply side tubing 112, and an upstream connector 126 (upstream relative to a drainage receptacle 120, where this connector may also or instead be a Vario connector by way of example) may be positioned on the drain side tubing 122. These connectors 116, 126 may allow for potential disconnection and/or reconfiguration of the tubing, providing flexibility in the system's setup.

The patient tubing 102 may terminate at a patient connector 106, which may be any as known in the art. In general, the patient connector 106 may be structurally configured to interface with a patient for a dialysis treatment. In certain implementations, an accessory 108 (such as a clamp, valve, flow restrictor, or the like) may be positioned on tubing in the system 100, e.g., to provide, promote, and/or control fluid flow in the system 100. The drain side of the system 100 may include drain side tubing 122 extending from a Y-connector 104 or otherwise connected to the patient tubing 102 for receiving effluent from the patient and transporting that effluent to a drainage receptacle.

Thus, the example peritoneal dialysis system 100 may demonstrate an interconnected arrangement of supply and drain components. This configuration may facilitate the flow of dialysis solution from supply bags to the patient, and the subsequent drainage of effluent from the patient to drain bags, enabling a peritoneal dialysis treatment.

It will be understood that the supply side tubing 112—e.g., that shown in FIGS. 1A-1D or as otherwise described herein—may generally include tubing and/or conduits in a peritoneal dialysis system or similar that are structurally configured to fluidically connect a supply of fresh dialysis solution to a patient's peritoneum. This may include, for example, tubing that extends from solution bags 130 to a Y-connector 104 or other junction point in the system 100. It will be understood that the drain side tubing 122—e.g., that shown in FIGS. 1A-1D or as otherwise described herein—may generally include tubing and/or conduits in a peritoneal dialysis system or similar that are structurally configured to fluidically connect a patient's peritoneum to a drainage receptacle 120 for collecting effluent. This may include, for example, tubing that extends from a Y-connector 104 or other junction point to drain bags 130 or other waste collection containers.

As stated above, FIG. 1A shows bags 130 aligned for connection (e.g., via bag connectors 134) to supply side connectors 114, and FIG. 1B shows the bags 130 so connected, but where the downstream connector 116 is disengaged, thereby separating the supply side tubing 112 from the patient tubing 102 and/or drain side tubing 122. In this manner, in this example system 100, the bags 130 may be coupled (e.g., via a Vario connector or similar) to the drain side tubing 122, e.g., using the upstream connector, where the bags 130 can thus become the drainage receptacle 120 in the system 100 during a draining of effluent from the patient connector 106 and patient tubing 102. FIG. 1C shows this connection, where the bags 130 are drainage receptacles 120 via connection of tubing extending therefrom to drain side tubing 122 using the upstream connector 126.

Thus, in some aspects, as shown in FIG. 1C, the bags 130 may maintain an engagement between bag connectors 134 and certain tubing connectors (e.g., drain side connectors 124 as shown in FIG. 1C, but where these connectors may be the same as the aforementioned supply side connectors 114 in FIG. 1A), e.g., where the entire bag/tubing assembly can be connected to a drain line via a single connector such as a Vario connector or the like.

In this manner, the peritoneal dialysis system 100 may include supply side tubing 112 structurally configured to fluidically connect a supply of dialysis solution 110 to a peritoneum of a patient via a patient connector 106. The supply of dialysis solution 110 may include one or more bags 130 containing fresh dialysis solution connected to the supply side tubing 112. And, in this manner, the peritoneal dialysis system 100 may include drain side tubing 122 structurally configured to fluidically connect the peritoneum of the patient to a drainage receptacle 120, which may include one or more bags 130—for example, the empty bags that were previously used (and drained of fresh dialysate supplied to the patient) as the supply of dialysis solution 110 in the system 100. The example peritoneal dialysis system 100 thus shows an interconnected arrangement of supply side tubing 112, patient tubing 102, and drain side tubing 122. This configuration may enable the transfer of fresh dialysis solution from the bags 130 to the patient's peritoneum, and subsequently, the transfer of effluent from the patient's peritoneum to the bags 130, which may have been relocated after being emptied.

However, as shown in FIG. 1D, problems would arise if the bags used as drainage receptacles 120 were then reconnected to the supply line for use as a supply of dialysate. Stated otherwise, using effluent as a supply of dialysate can be problematic. Thus, the present teachings include techniques to prevent the reuse of bags 130 as a supply of dialysis solution 110 when those bags 130 have already been used as a drainage receptacle 120 and are thus filled with effluent and not fresh dialysate. In general, the present teachings may include two general solutions—(i) an indicator (e.g., visual indicator) for the bags, and (ii) mechanical arrangements to prevent reconnection of used bags as a supply of dialysate—where it will be understood that these general solutions may be used in isolation or combination in dialysis systems such as that described with reference to FIGS. 1A-1D or similar.

### Indicator for Bags

The present teachings may include components in or on a bag intended for use as both a supply of dialysate and a drainage receptable to prevent reuse of the bag as the supply after it has already been used as the drainage receptacle. Thus, the bag itself may include one or more features to prevent users from connecting a used solution bag filled with effluent to a fresh, sterile tubing system for a subsequent treatment.

In certain aspects, the present teachings include the use of a sensor, such as a chemical sensor. For example, a chemical sensor or the like may be disposed inside of a solution bag. By way of example, the sensor can include a strip of material and/or coating that reacts with a chemical in effluent but is non-reactive with fresh dialysate—e.g., the sensor may detect urea, creatinine, or the like. In this manner, when waste drained from a patient into an empty solution bag makes contact with such a sensor, this may cause it to provide a signal to the user that the bag is only suitable as a drain bag and not a supply of fresh dialysate. By way of example, such a signal could include a color change, the appearance of symbols and/or text on the bag, a change in opacity, and the like. Other alerts in addition to or instead of visual indicators are also or instead possible.

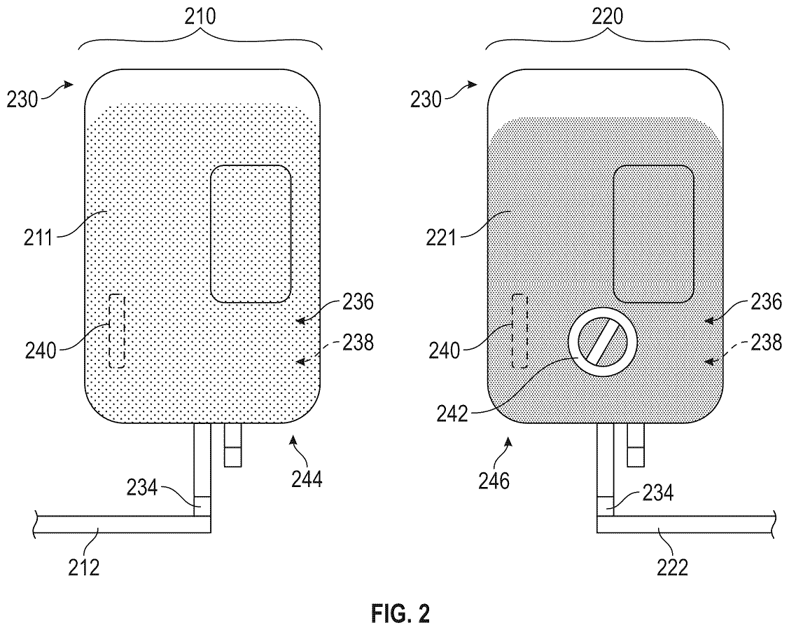

FIG. 2 shows a bag in two different visual states, according to a representative example. In this manner, FIG. 2 shows a side-by-side comparison of two states of a bag 230, e.g., a peritoneal dialysis bag. Specifically, FIG. 2 shows a supply of dialysis solution 210, where the bag 230 includes fresh dialysis solution 211 therein and is in a first visual state 244, and FIG. 2 shows a drainage receptacle 220, where the bag 230 includes effluent 221 therein and is in a second visual state 246.

The bag 230 may be part of a peritoneal dialysis system, such as the system 100 of FIGS. 1A-1D, or others known in the art or described herein. Such a system may include supply side tubing 212 structurally configured to fluidically connect a supply of dialysis solution 210 to a peritoneum of a patient, and drain side tubing 222 structurally configured to fluidically connect the peritoneum of the patient to a drainage receptacle 220. In this manner, the bag 230 may be engaged with one or more connectors 234 structurally configured to independently connect, directly or indirectly, with each of supply side tubing 212 and drain side tubing 222. In this manner, the bag 230, when filled with fresh dialysis solution 211 and connected with the supply side tubing 212, may serve as the supply of dialysis solution 210 in the system—this state may be represented by the left side of FIG. 2. Further, the bag 230, when emptied and connected with the drain side tubing 222, may function as the drainage receptacle 220 to receive effluent 221 in the system—this state may be represented by the right side of FIG. 2. This dual functionality may be facilitated by the connector 234, and/or components (e.g., other tubing and connectors) coupled thereto, that allows the bag 230 to be independently connected to either the supply side tubing 212 or the drain side tubing 222, depending on the current stage of a dialysis treatment and/or the contents (or lack thereof) of the bag 230.

The fresh dialysis solution 211—e.g., as described with reference to FIG. 2 or any of the other devices, systems, and techniques described herein—may include a sterile aqueous solution containing various solutes in specific concentrations suitable for peritoneal dialysis or similar. In some aspects, the fresh dialysis solution 211 may include electrolytes such as sodium, calcium, magnesium, chloride ions, and the like. The solution may also contain a buffer system, which in some cases may include one or more of lactate, bicarbonate, and similar. In some implementations, the fresh dialysis solution 211 may include an osmotic agent, e.g., icodextrin, amino acids, or other suitable osmotic materials. The fresh dialysis solution 211 may have a pH in a physiologically compatible range. In some aspects, the solution may be packaged in sterile containers and may appear clear and colorless. The solution may be free of particulate matter and may demonstrate a specific gravity and osmolality within predetermined ranges suitable for peritoneal dialysis. In some cases, the fresh dialysis solution 211 may include additives such as antibiotics, heparin, or other therapeutic agents.

Effluent 221—e.g., as described with reference to FIG. 2 or any of the other devices, systems, and techniques described herein—may include any fluid drained from a patient's peritoneum during/after a dialysis treatment. The effluent 221 may thus contain various substances removed from the patient's blood during a dialysis treatment. These substances may include metabolic waste products, excess electrolytes, and other compounds that the kidneys would typically filter out in healthy individuals. The composition of effluent 221 may include urea, which may be produced during protein metabolism. Creatinine may also be present in the effluent 221 as a byproduct of muscle metabolism. In some implementations, the effluent 221 may contain elevated levels of electrolytes such as sodium, potassium, calcium, and/or phosphate that have been removed from the patient's blood. The effluent 221 may also or instead contain metabolic acid products that accumulate in patients with kidney failure. In some cases, trace elements such as aluminum, copper, and zinc may be present in the effluent 221. The effluent 221 may additionally contain medications or their metabolites that have been cleared from the patient's bloodstream during dialysis. In certain aspects, the effluent 221 may include inflammatory markers or proteins that have passed through the peritoneal membrane during treatment.

The bag 230 may include a sensor 240 in communication with an interior thereof—e.g., disposed within a void 238 of the bag 230 so that it can engage with contents of the bag 230. The sensor 240 may be reactive with, and/or responsive to, one or more contents of the effluent 221 but non-reactive with the fresh dialysis solution 211. The sensor 240 may include, or be in communication with, a visual indicator 242 that changes from a first visual state 244 to a second visual state 246 when the sensor reacts with (and/or detects or otherwise responds to) one or more contents of the effluent 221 to visually indicate that the bag 230 contains waste for preventing subsequent use of the bag 230 as the supply of dialysis solution 210 in a dialysis treatment system. For example, and as illustrated, in the first visual state 244 the visual indicator 242 may be substantially invisible, whereas in the second visual state 246 (after reaction of the sensor 240) the visual indicator 242 may be visible.

The sensor 240 may be a chemical sensor. That is, the sensor 240 may include a device and/or material capable of detecting or reacting with one or more chemical components present in peritoneal dialysis effluent 221 but that are not present in fresh dialysis solution 211. In certain implementations, a chemical sensor may include a strip of material or coating applied to an interior surface of the bag 230. The chemical sensor 240 may be configured to react specifically with chemicals typically present in peritoneal dialysis effluent, such as urea, creatinine, and/or other waste products. Examples may include, but are not limited to, litmus paper, biosensors, electrochemical sensors, or potentiometric sensors that can detect substances such as urea, creatinine, electrolytes, metabolic acid products, and/or other waste components typically found in dialysis effluent 221.

In some aspects, the sensor 240 may include a colorimetric indicator that changes color when exposed to urea, which may be present in effluent 221 at concentrations of about 300-400 mg/dL but absent in fresh dialysate. In some aspects, the sensor 240 may incorporate urease enzymes coupled with pH indicators that produce a visible color change when urea is metabolized. In some implementations, the sensor 240 may include creatinine-sensitive materials that undergo a color shift when exposed to creatinine levels typical in effluent 221 (e.g., in amounts of about 5-15 mg/dL) while remaining stable in fresh dialysate. The sensor 240 may utilize picric acid or similar compounds that form colored complexes specifically with creatinine. The sensor 240 may incorporate potentiometric elements sensitive to changes in electrolyte concentrations and/or otherwise be configured to detect a threshold amount of electrolytes or similar substances. In some cases, ion-selective electrodes may detect elevated potassium, calcium, or phosphate levels characteristic of effluent 221 without responding to the controlled electrolyte content of fresh dialysate. In some aspects, the sensor 240 may include oxidation-reduction indicators that react with metabolic waste products present in effluent 221, where these indicators may change color when exposed to organic acids, ketones, or other metabolites absent from fresh dialysate. The sensor 240 may utilize protein-sensitive dyes that respond to albumin or other proteins that may pass into the effluent 221. In some implementations, the sensor 240 may incorporate bromocresol green or similar indicators that produce visible color changes when bound to proteins while remaining unchanged in protein-free fresh dialysate. In some cases, the sensor 240 may include pH-sensitive compounds that respond to the typically more acidic pH of effluent 221 compared to fresh dialysate. The sensor 240 may utilize combinations of indicators that provide distinct color changes across relevant pH ranges. The sensor 240 may incorporate inflammatory markers that react with cytokines or other inflammation-associated molecules present in effluent 221. In some aspects, these markers may include antibody-based detection systems or synthetic molecules designed to bind specifically to inflammatory mediators. In some implementations, the sensor 240 may include metal ion indicators that respond to elevated levels of trace metals like copper or zinc in effluent. These indicators may form colored complexes with metal ions while remaining colorless in fresh dialysate containing minimal metal content.

The sensor 240 may also or instead include a biosensor having biological recognition elements to detect specific molecules present in effluent 221. In some aspects, a biosensor may include immobilized enzymes such as urease that catalyze reactions with waste products. The enzymatic reaction may generate products that can be detected through changes in pH, conductivity, and/or optical properties. In some implementations, the biosensor may utilize antibodies specific to proteins or inflammatory markers found in effluent 221. The antibodies may be conjugated to reporter molecules that produce visible signals when binding occurs. The biosensor may include multiple recognition elements to detect different effluent components simultaneously.

The sensor 240 may also or instead include an electrochemical sensor that measures changes in electrical properties when exposed to effluent contents. In some aspects, the sensor 240 may include electrodes that detect variations in conductivity between fresh dialysate and effluent 221. The sensor 240 may measure impedance changes resulting from the presence of ions and organic molecules in effluent 221. In some cases, the electrochemical sensor may utilize ion-selective electrodes to detect specific electrolytes. The electrodes may generate voltage signals proportional to ion concentrations, allowing detection of elevated levels of potassium, calcium, or other ions in effluent 221 compared to fresh dialysate. The electrochemical sensor may incorporate amperometric detection methods. In some implementations, the sensor may measure current generated by oxidation or reduction of electroactive species present in effluent 221. The sensor 240 may include working electrodes modified with catalytic materials to enhance sensitivity to specific analytes.

In some aspects, the sensor 240 may combine multiple detection modes. By way of example, the sensor 240 may include arrays of electrodes for simultaneous measurement of different parameters such as pH, conductivity, and specific ion concentrations. The sensor 240 may be designed to provide stable measurements in the presence of proteins and other potential interfering substances in effluent 221. Other sensors 240 are also or instead possible.

The bag 230 may also include a visual indicator 242 in communication with the sensor 240 (e.g., integral with the sensor 240). The visual indicator 242 may include a visible change or signal that can be observed by a user to indicate a change in a state or condition of the bag 230—e.g., a change in the contents of the bag 230. In the context of peritoneal dialysis bags, this may include, for example, a color change on a portion of the bag's surface and/or a color change in the appearance of contents of the bag 230, the appearance of a symbol and/or text, a change in opacity of the bag 230 and/or contents thereof, and/or any other visually detectable alteration that signifies the bag 230 contains effluent 221 rather than fresh dialysis solution 211.

In general, the visual indicator 242 may be in a first visual state 244 when the bag 230 contains fresh dialysis solution 211 as represented by the supply of dialysis solution 210 shown on the left side of FIG. 2. The first visual state 244 may include an absence of any visible indication or in the appearance of the bag's contents. The first visual state 244 may also or instead include other visual features, such as a symbol or text visible on the bag 230 (e.g., text such as “Clean,” “Fresh,” “Okay to use,” or a symbol such as a happy face, checkmark, or the like, and so forth).

As the bag transitions from containing fresh dialysis solution 211, to being emptied, to containing effluent 221, the sensor 240 may detect and/or react with contents of the effluent 221. This reaction may trigger a change in the visual indicator 242 from the first visual state 244 to a second visual state 246, as represented by the drainage receptable 220 shown on the right side of FIG. 2. The second visual state 246 may include the presence of a visible indication on the bag's surface and/or a change in the appearance of the bag's contents.

In some aspects, the first visual state 244 includes an absence of visual indication, and the second visual state 246 includes a presence of a visual indication—e.g., the visual indicator 242 shown on the right side of FIG. 2 in the form of a universal “don't” or “no” or “stop” symbol. In some implementations, the visual indicator 242 may change color, display a symbol, or alter the opacity of the bag 230 and/or its contents in response to the sensor 240 detecting effluent 221. For example, a visual indicator 242 in the form of a symbol may appear on the exterior surface 236 of the bag 230 when the sensor 240 reacts with or otherwise detects effluent contents, providing a clear visual cue that the bag 230 contains waste rather than fresh dialysis solution.

The second visual state 246 may also or instead include a color change from the first visual state 244. Such a color change may affect a portion of a surface of the bag 230. For example, the color change may form one or more of a symbol and text visible on the surface of the bag 230, e.g., the exterior surface 236 thereof. The color change may also or instead affect the appearance of contents of the bag 230. For example, the contents of the bag 230 may become more opaque, or change to a different color, in response to the sensor 240 detecting effluent 221. By way of further example, in some aspects, the visual indicator 242 may undergo a color change that affects a portion of the exterior surface 236 of the bag 230. This color change may be localized to a specific area on the bag's surface. For example, a section of the bag's surface may transition from a transparent or neutral color in the first visual state 244 to a distinct color, such as red or yellow, in the second visual state 246.

The visual indicator 242 may also or instead include one or more of a color, a symbol, text, patterning, an image, and the like. For example, the visual indicator 242 may, in some aspects, cause words to appear on the bag 230 when triggered by the sensor 240. By way of further example, text such as “Effluent” or “Waste” or “Do Not Reuse” may become visible on the bag's surface when the sensor 240 detects one or more contents of effluent 221.

The visual changes facilitated by the interaction between the sensor 240 and the visual indicator 242 may provide clear and unmistakable signals to users about the current contents of the bag 230. These visual cues may help prevent accidental reuse of bags 230 containing effluent 221 as a source of fresh dialysis solution 210, thereby enhancing patient safety in peritoneal dialysis treatments.

In some implementations, the changes facilitated by the interaction between the sensor 240 and the visual indicator 242 may also or instead include non-visual features or elements. In this manner, the visual indicator 242 may instead be a tactile indicator, an audio indicator, a communications interface, or similar. For example, such an indicator may provide a texture change to an exterior surface 236 of the bag 230, e.g., creating bumps or other discernable tactile features. Also or instead, the sensor 240 may communicate with a device having audio capabilities (e.g., a portion of a dialysis system) that plays a sound or alert when effluent 221 is detected and/or when a used bag is attempted to be connected as a supply of fresh dialysate. Such non-visual indicators may be advantageous for users that are visually impaired.

The bag 230 shown in this figure, or any of the bags described herein, may include one or more structural components designed to facilitate its use in a peritoneal dialysis system, such as any of the systems described herein or otherwise known in the art. In some aspects, the bag 230 may include an exterior surface 236 that provides a protective barrier for the contents of the bag 230 and may serve as a substrate for visual indicators or other markings as described herein. The bag 230 may also define a void 238 in an interior of the bag 230, which may be structurally configured to be at least partially filled with a fluid. In some cases, the void 238 may be designed to accommodate a specific volume of fluid, such as a standard amount of dialysis solution and/or effluent. In certain implementations, the bag 230 may include one or more connectors 234 in fluid communication with the void 238. These connectors 234 may be any as described herein or otherwise known in the art. One or more of the connectors 234 may be structurally configured to independently connect with each of supply side tubing 212 and drain side tubing 222 in a peritoneal dialysis system. This dual connectivity capability may allow the bag 230 to serve multiple functions within a dialysis system.

For example, at least one of the connectors 234 may be structurally configured to independently connect with supply side tubing 212 fluidically connecting a supply of dialysis solution 210 to a peritoneum of a patient. When filled with fresh dialysis solution 211 and connected with the supply side tubing 212, the bag 230 may be configured to be the supply of dialysis solution 210 in a peritoneal dialysis system. The same connector 234, or another connector (e.g., a connector downstream from the connector 234), may be structurally configured to independently connect with drain side tubing 222 fluidically connecting the peritoneum of the patient to a drainage receptacle 220. When emptied and connected with the drain side tubing 222, the bag 230 may be configured to be the drainage receptacle 220 to receive effluent 221 in a peritoneal dialysis system. The ability of the bag 230 to connect with both supply side tubing 212 and drain side tubing 222 may allow for efficient use of resources in a peritoneal dialysis system. In some cases, this dual functionality may reduce the number of separate bags 230 required for a dialysis treatment, potentially simplifying the procedure for patients and caregivers.

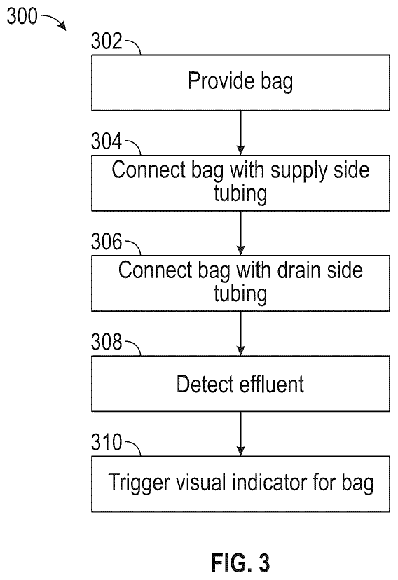

FIG. 3 is a flow chart of a method of identifying a drainage receptacle for dialysis, according to a representative example. The method 300 may be performed using any of the devices and systems described herein—e.g., the system 100 of FIGS. 1A-1D and/or the bags 230 of FIG. 2. In general, the method 300 may comprise several steps designed to facilitate the safe and effective use of bags in a peritoneal dialysis system.

As shown in step 302, the method 300 may include providing a bag structurally configured to connect—directly or indirectly—with each of supply side tubing and drain side tubing in a peritoneal dialysis system. The bag may be any as described herein, and for example may include fresh dialysis solution, a sensor (e.g., a chemical sensor) in an interior thereof, and a visual indicator (or other indicator, such as audio and/or tactile) in communication with the sensor. In certain implementations, this includes a visual indicator that is present in a first visual state when the bag contains the fresh dialysis solution.

As shown in step 304, the method 300 may include fluidically connecting the bag with the supply side tubing and supplying the fresh dialysis solution from the bag to a peritoneum of a patient during a first sequence of operation in a dialysis procedure. This step 304 may result in substantially emptying the bag of the fresh dialysis solution.

Following supplying fresh dialysis solution, the method 300 may move to step 306, where the now substantially emptied bag can be reused as a waste receptacle in a dialysis system. That is, as shown in step 306, the method 300 may include fluidically connecting the bag with the drain side tubing and receiving effluent from the peritoneum of the patient during a second sequence of operation in the dialysis procedure. This step 306 may result in substantially filling the bag with effluent.

As shown by step 308, the method 300 may include detecting, via the sensor, one or more contents of the effluent. For example, a chemical sensor may be reactive with specific components typically found in peritoneal dialysis effluent, such as urea, creatinine, and/or other waste products.

As shown by step 310, the method 300 may include triggering a change in the visual indicator from the first visual state to a second visual state to visually indicate that the bag contains effluent. This change in visual state may serve as a clear signal to users that the bag now contains waste and should not be used as a source of fresh dialysis solution. This step 310 may also or instead include triggering other non-visual changes and/or alerts, such as transmitting a signal to send an alert (e.g., an audio alert, a notification, etc.) and/or affecting a tactile change to the bag and/or a surface thereof.

The method 300 may thus provide a systematic approach to using bags in a peritoneal dialysis system, transitioning them from containing fresh dialysis solution to serving as drainage receptacles, while incorporating safeguards to prevent the accidental reuse of effluent-filled bags as a source of fresh dialysis solution. This method 300 may enhance patient safety, particularly in home dialysis settings or for users with cognitive impairments, by providing clear visual cues against the misuse of dialysis bags.

The present teachings may thus include bags for a peritoneal dialysis system, the bag including: an exterior surface; a void in an interior of the bag structurally configured to be filled with a fluid; and one or more connectors in fluid communication with the void, at least one of the one or more connectors structurally configured to independently connect, directly or indirectly, with each of (i) supply side tubing fluidically connecting a supply of dialysis solution to a peritoneum of a patient, and (ii) drain side tubing fluidically connecting the peritoneum of the patient to a drainage receptacle, where the bag, when filled with fresh dialysis solution and connected with the supply side tubing, is configured to be the supply of dialysis solution in a peritoneal dialysis system, and where the bag, when emptied and connected with the drain side tubing, is configured to be the drainage receptacle to receive effluent in the peritoneal dialysis system. The bag may further include a chemical sensor (or other sensor) in the interior of the bag, the chemical sensor reactive with one or more contents of the effluent but non-reactive with the fresh dialysis solution, and an indicator (e.g., a visual indicator) responsive to the chemical sensor, the indicator configured to change from a first state to a second state when the chemical sensor reacts with the one or more contents of the effluent to indicate that the bag contains waste for preventing subsequent use of the bag as the supply of dialysis solution in the peritoneal dialysis system.

### Mechanical Techniques to Prevent Reuse of Effluent Bags

In addition to or instead of sensors in a bag that can be used to detect effluent and provide an alert (e.g., a visual, audio, and/or tactile indicator) for users to prevent reuse of the effluent-filled bag as a supply of fresh dialysate in a dialysis system, the present teachings may include mechanical techniques to prevent the reuse of effluent bags as a dialysate supply in a dialysis system. That is, the present teachings may also or instead include peritoneal dialysis systems with mechanical safeguards to prevent accidental reuse of effluent-filled bags as a source of fresh dialysis solution. In some aspects, these safeguards may include connectors that become mechanically altered and/or permanently coupled upon first use, preventing subsequent reconnection to the supply side of a system. These techniques may incorporate breakpoints, one-way locking mechanisms, and/or other features that render the bag connectors incompatible with supply side tubing after initial use. These physical barriers against improper reuse may enhance patient safety, particularly for users with cognitive impairments or in settings with limited oversight, through the mechanical prevention of potentially harmful connections.

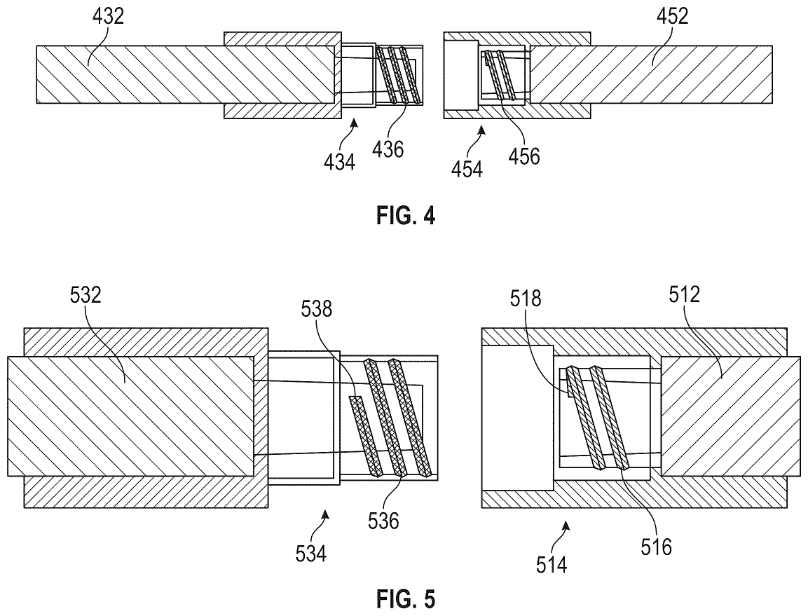

FIG. 4 shows a typical bag and tubing connector. That is, FIG. 4 represents connectors that may be found in the art, where the connectors are shown for clarity and understanding of the present teachings, features of which are shown by way of example in FIGS. 5-12. Thus, the connectors of FIG. 4 and their respective features may be arranged to create connections between components in a typical peritoneal dialysis system. In some cases, the features of these connectors may allow for repeated connections and disconnections between components. This design may not inherently prevent the reconnection of bags to the supply side of the dialysis system after they have been used for effluent collection. Thus, the present teachings may improve upon one or more of the connectors shown in this figure. The connectors in a dialysis system may include a bag connector 434 and a system connector 454.

The bag connector 434 may be engaged with bag tubing 432 that leads to a bag—e.g., a bag containing fresh dialysate, where the bag is intended to be used as the supply of fresh dialysis solution in a dialysis system, or a substantially empty bag that is intended to be used as a drainage receptable to collect effluent in a dialysis system. The bag connector 434 may be any type known in the art, and may include one or more engagement features—e.g., the first engagement portion 436 as shown in the figure—to facilitate engagement with connectors and/or tubing in a dialysis system or similar. That is, the first engagement portion 436 may be structurally configured to couple with the second engagement portion 456 of a system connector 454. By way of example, the first engagement portion 436 may include threading or the like, where the second engagement portion 456 includes cooperating threading or the like. Also or instead, the bag connector 434 (or other connectors described herein, with reference to FIG. 4 or otherwise) may include one or more of: Luer lock fittings, which provide a secure, leak-proof connection through a threaded locking mechanism, and which may have male and female components that twist together to create a tight seal; quick-connect fittings, which may incorporate a push-to-connect mechanism with an internal locking feature that engages when the tubing is inserted; barbed connectors, which may have a tapered, ridged end that inserts into flexible tubing, creating a tight seal as the tubing stretches over the barbs; clamp connectors, which may feature a hinged mechanism that closes around the tubing, securing it in place and providing a seal when engaged; bayonet-style connectors, which involve a twisting motion to lock the connection, and which may have interlocking tabs or pins that secure the connection when rotated into place; specialized connectors designed for specific components, such as Y-connectors for joining multiple tubes or transfer set connectors for interfacing with a patient's catheter; and/or the like. It is to be understood that features described with respect to the first engagement portion 436 may instead be present on the second engagement portion 456, and vice versa, and may in such configuration result in the same or equivalent function. The connectors described herein may be made from materials compatible with medical applications, such as medical-grade plastics or stainless steel. In some implementations, the connectors may incorporate features like color coding or unique shapes to prevent misconnections between different parts of the system.

The system connector 454 may be engaged with system tubing 452 that leads to a component or portion of a dialysis system—e.g., the system tubing 452 may fluidically connect, directly or indirectly, with a patient's peritoneum when the bag is intended to be used as the supply of fresh dialysis solution in a dialysis system, or the system tubing 452 may fluidically connect, directly or indirectly, with drainage tubing and/or a drainage receptacle. The system connector 454 may be any type known in the art, and may include one or more engagement features—e.g., the second engagement portion 456 as shown in the figure—to facilitate engagement with connectors and/or tubing in a dialysis system or similar. That is, the second engagement portion 456 may be structurally configured to couple with the first engagement portion 436 of a bag connector 434. In this manner, it will be understood that the system connector 454 and/or the second engagement portion 456 may include any of the features described above with respect to the bag connector 434 and/or the first engagement portion 436.

It will be understood that the bag connector 434 and/or the first engagement portion 436, and/or the system connector 454 and/or the second engagement portion 456, may be adapted using techniques of the present teachings to mitigate or prevent the later use of a bag that has been used as a drainage receptacle as a supply of dialysate in a dialysis system.

FIG. 5 shows a first connector and a supply side connector, according to a representative example. Specifically, FIG. 5 shows a first connector 534 and a supply side connector 514 that may be part of a peritoneal dialysis system or similar, where the system includes one or more mechanical features to prevent reuse of a bag previously used as a drainage receptacle for effluent as a supply of dialysis solution in the system. The system may be any as described herein, e.g., the system 100 of FIGS. 1A-1D. In this manner, such a system will be understood to optionally include supply side tubing 512 with the supply side connector 514 structurally configured to fluidically connect to a supply of dialysis solution (e.g., a bag, such as any as described herein) to transfer the dialysis solution to a peritoneum of a patient. The system may further include drain side tubing with a drain side connector structurally configured to fluidically connect to a drainage receptacle to transfer effluent from the peritoneum of the patient to the drainage receptacle. It will be understood that although FIG. 5 is showing supply side tubing 512 and a supply side connector 514, the features of the present teachings described herein may also or instead be present on drain side tubing and a drain side connector.

The system featuring the first connector 534 and the supply side connector 514 may further include a bag, which may be any as described herein—e.g., it may include any of the features described herein, such as with reference to FIG. 2. In this manner, the bag may include the first connector 534—e.g., the bag tubing 532 shown in the figure may fluidically connect, directly or indirectly, an interior of the bag with the first connector 534.

In general, the first connector 534 may be structurally configured such that the bag is couplable only once to the supply side connector 514. In this manner, the bag, when filled with fresh dialysis solution, may be configured to be the supply of dialysis solution in the system—but only once. The bag may further be structurally configured for fluidically coupling to a drain side connector—directly or indirectly—after being coupled to the supply side connector 514. In this manner, the bag, when emptied, may be configured to be the drainage receptacle for effluent in the system, but where the bag thereafter cannot subsequently be reconnected to the supply side tubing 512.

There are a plurality of ways that the present teachings may prevent or mitigate against reuse of a bag on the supply side of a system when the bag has been used as a drainage receptacle for effluent in the system. For example, in certain aspects, the first connector 534 and the supply side connector 514 may permanently engage, thereby preventing the first connector 534 from subsequently reengaging with another supply side connector 514—but, by way of example, where a downstream connector facilitates coupling of the tubing structure created by the combination of the first connector 534 and the supply side connector 514. In other aspects, the first connector 534 may be mechanically altered (e.g., by the supply side connector 514) such that it cannot be reengaged therewith and/or with other supply side connectors 514. Also or instead, the tubing structure created by the combination of the first connector 534 and another connector may create a breakpoint or the like, which can allow for transport of a bag engaged with the first connector 534 (e.g., for disposal after use as a drainage receptacle) but that can prevent reengagement with a supply side connector 514. The configuration of a first engagement portion 536 of the first connector 534 and/or a second engagement portion 516 of the supply side connector 514 may aid in the prevention of reengagement of a bag used as an effluent collector with the supply side in a dialysis system.

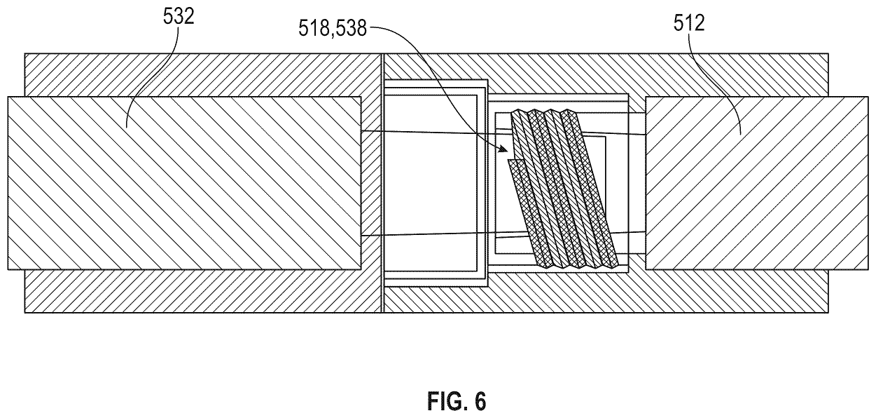

For example—as shown in FIGS. 5 and 6, where FIG. 6 shows the first connector 534 engaged with the supply side connector 514—the first connector 534, when engaged with the supply side connector 514, may become permanently coupled to the supply side connector 514. Specifically, the first connector 534 may include a first feature 538 and the supply side connector 514 may include a second feature 518 that engage to form a permanent coupling. That is, the first feature 538 and the second feature 518 may be configured to engage such that, when the first connector 534 and the supply side connector 514 are fully seated relative to one another (or substantially fully seated), the first feature 538 and the second feature 518 permanently lock engagement therebetween. By way of example, the first feature 538 may include a notch or the like, and the second feature 518 may include a cooperating catch or the like. In some instances, the catch is included on an angled barb or the like.

In such aspects featuring a permanent engagement of the first connector 534 and a supply side connector 514 (or another connector of a dialysis system), a system may further include a downstream connector couplable to other tubing, connectors, and the like. For example, such a downstream connector may be couplable, directly or indirectly, to the drain side connector for use of the bag, when emptied, as a drainage receptacle in the system. By way of example, such a downstream connector may include any as described with reference to one or more of the downstream connector 116 and the upstream connector 126 of FIGS. 1A-1D.

It will be understood that the first engagement portion 536 and the second engagement portion 516 are shown as cooperating threading in these and other figures herein, although other cooperating engagement portions are also or instead possible, including without limitation: barbed fitting engagements, push-to-connect fitting engagements, Luer fitting engagements, compression fitting engagements, cam and groove fitting engagements, and the like, where any of which may be adapted for use with the present teachings without departing from the scope of this disclosure.

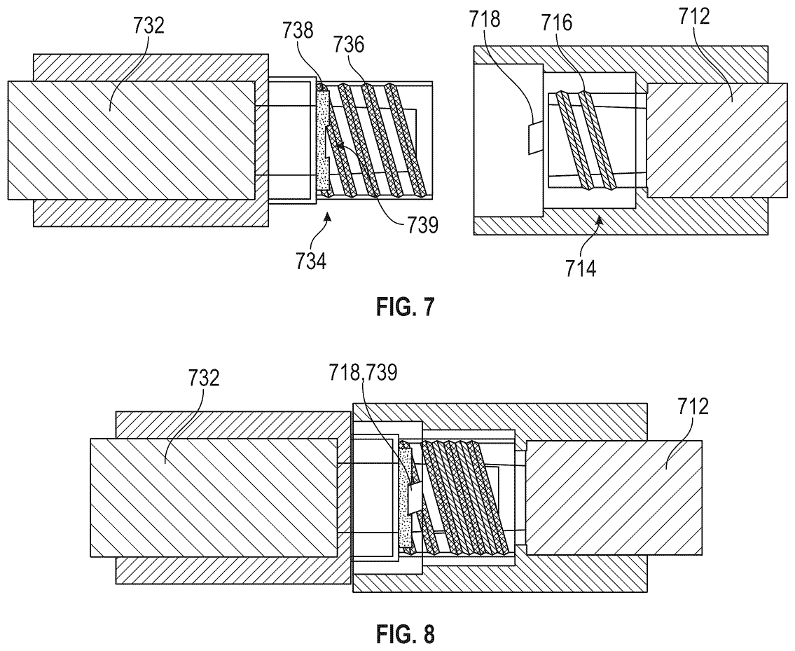

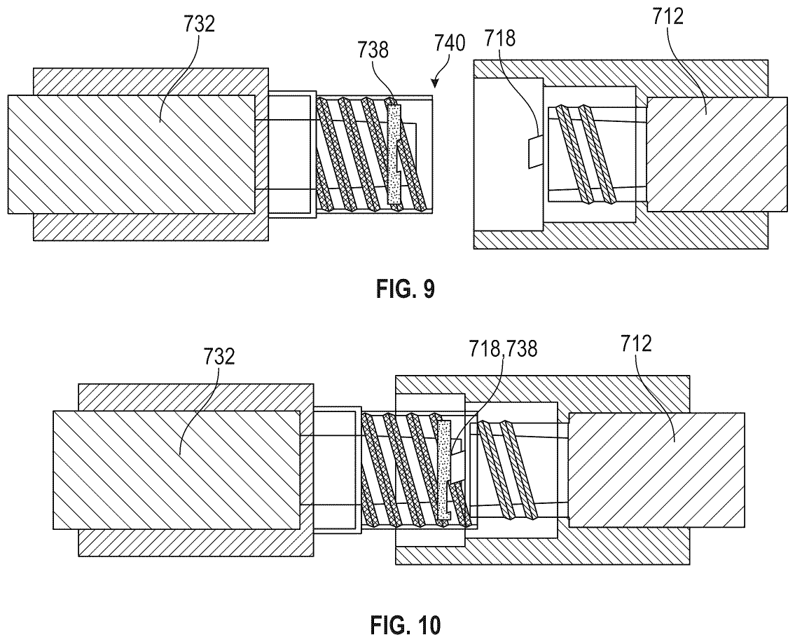

FIG. 7 shows a first connector and a supply side connector according to a representative example; FIG. 8 shows a first connector engaged with a supply side connector according to a representative example; FIG. 9 shows a first connector and a supply side connector according to a representative example; and FIG. 10 shows a first connector being prevented from engagement with a supply side connector according to a representative example. That is, FIGS. 7-10 may represent a sequence of operations demonstrating how certain aspects of the present teachings may be used to prevent reengagement of a bag used as an effluent collector with the supply side in a dialysis system. That is, these figures show a first connector 734 engaged with bag tubing 732 being mechanically altered by a supply side connector 714 engaged with supply side tubing 712 such that reengagement is prevented (or impeded) thereafter—see, e.g., FIG. 10.

As shown in FIG. 7, the first connector 734 may include a first engagement portion 736, which may be any as described herein—e.g., threading or the like. The first connector 734 may also or instead include a first feature 738, which may include a notch 739 or the like. As further shown in FIG. 7, the supply side connector 714 may include a second engagement portion 716, which may be any as described herein—e.g., threading or the like. The supply side connector 714 may also or instead include a second feature 718, which may include a protrusion or the like. Other configurations are also or instead possible.

In general, and as described above, these or other similar features may provide for the first connector 734 being mechanically altered upon engagement with the supply side connector 714 such that, upon disengagement from the supply side connector 714, the first connector 734 cannot be reengaged with the supply side connector 714 (the same supply side connector 714 or another supply side connector 714). By way of example, the first connector 734 may include a first feature 738 and the supply side connector 714 may include a second feature 718, where the second feature 718 is structurally configured to engage with the first feature 738 and to move the first feature 738 from an initial position (e.g., shown in FIGS. 7 and 8) to a final position (e.g., shown in FIGS. 9 and 10) through one or more of an engagement operation (e.g., screwing the connectors together or otherwise coupling the connectors) and a disengagement operation (e.g., unscrewing the connectors or otherwise decoupling the connectors). The final position of the first feature 738 may prevent reengagement between the first connector 734 and the supply side connector 714.

More specifically, by way of example, the first feature 738 may include a ring with a notch 739, and the second feature 718 may include a tab (or other protrusion or the like). In this specific example, the tab of the second feature 718 may be engageable with the notch 739 when the first connector 734 and the supply side connector 714 are fully seated relative to one another. In this manner, when the supply side connector 714 is disengaged from the first connector 734, the tab may move the ring outward towards an opening 740 in the first connector 734 thereby at least partially blocking the opening from receiving the supply side connector 714, e.g., as shown in FIG. 10. Said another way, in the initial position, the first feature 738 may be arranged to engage with the second feature 718, while in the final position, the first feature 738 may be arranged to resist or prevent engagement with the second feature 718, and may further, or thereby, be arranged to resist or prevent engagement of the first connector 734 with the supply side connector 714.

Thus, in some aspects, the first feature 738 may be configured as a ring-like structure or the like that is movably positioned within the first connector 734. The ring-like structure may be dimensioned to fit within an internal chamber of the first connector 734 while maintaining the ability to translate along a central longitudinal axis of the first connector 734. In certain implementations, the first feature 738 may include alternative configurations such as a collar, sleeve, and/or annular member that encircles an internal passage of the first connector 734. The first feature 738 may also or instead be implemented as a segmented ring structure, a C-shaped element, and/or multiple discrete elements arranged circumferentially within the first connector 734. In some cases, the first feature 738 may include a spring-loaded mechanism and/or biasing element that influences its movement (and/or its features configured for engagement, or configured for preventing engagement) within the first connector 734.

The interaction between the first feature 738 and the second feature 718 may involve a specific directional movement pattern that occurs during engagement and/or disengagement operations. In particular, the first feature 738 may be configured to move along a length of the first connector 734 in a direction substantially parallel to a central axis of the first connector 734. During initial engagement, the first feature 738 may be positioned in a first axial position that is more proximal to a base or interior portion of the first connector 734, allowing the second feature 718 to engage with the notch 739 when in this substantially proximal position (see, e.g., FIG. 7, which shows the first feature 738 in its first axial position; and see, e.g., FIG. 8, which shows the second feature 718 engaged with the first feature 738—and more specifically a notch 739 thereof—when in this first axial position within the first connector 734). Upon disengagement of the connectors, the interaction between the second feature 718 and the notch 739 (and/or other similar engagements) may cause the first feature 738 to translate axially toward a second axial position that is more distal, positioning the first feature 738 closer to an opening of the first connector 734 (see, e.g., FIG. 9, which shows the first feature 738 in its second axial position). In this second axial position, the first feature 738 may prevent reengagement with the supply side connector 714—e.g., the first feature 738 may be prevented from moving back to the first axial position and/or the first feature 738 may be prevented from engagement with the second feature 718 when in this second axial position.

The axial movement of the first feature 738 may occur through rotational and/or sliding motions that may correspond to the threading engagement between the first connector 734 and the supply side connector 714. In some instances, the first feature 738 may initially be disposed in a first axial position within the first connector 734 that is more proximal to a base or interior portion of the first connector 734. In other instances, the first connector 734 may be moved from an initial position to the first axial position. By way of example, as the connectors are threaded together, the second feature 718 may engage with the notch 739 and guide the first feature 738 toward the proximal, first axial position. During the disengagement process, the rotational motion used to unthread the connectors may cause the second feature 718 to apply a force to the first feature 738 (e.g., through the notch 739), driving the first feature 738 in a distal direction along the central axis of the connectors. This axial displacement may position the first feature 738 in a second axial position that is more distal within the first connector 734. In this second axial position, the first feature may prevent subsequent engagement and/or insertion of the supply side connector 714 or similar connectors with the first connector 734. In some aspects, this is caused by the relative positioning of the notch 739 or similar; this may also or instead be caused by a locking of the first feature 738 in the second axial position; this may also or instead be caused the first feature 738 extending into or partially obstructing the opening 740 of the first connector 734, creating a physical barrier that prevents subsequent insertion of the supply side connector 714 or similar connectors; and other arrangements are also or instead possible without departing from the scope of this disclosure.

That is, in certain implementations, the first feature 738 (or the first connector 734 more generally) may include structural elements that enhance its blocking capability once positioned in the second axial position. For example, a ring-like structure may include radially extending tabs, flanges, or protrusions that become more prominent when the first feature 738 is in the second axial position. The first feature 738 may also or instead include a tapered or stepped configuration that provides different blocking characteristics depending on its axial position within the first connector 734. And, in some aspects, the movement of the first feature 738 may be irreversible once it reaches the distal position, ensuring that the blocking effect cannot be easily defeated by manipulation of the first connector 734.

Thus, in certain aspects, the present teachings may include placing a feature (e.g., an indent/notch or similar) within a bag connector and placing a cooperating feature (e.g., a protrusion or similar) on one or more other system connectors—e.g., on a Vario line connector. In this manner, once connected, these connectors can lock together such that they cannot be disconnected (see FIG. 6, for example). In some aspects, such an interface would be present only between the solution bags and fresh dialysate tubing connectors, where the tubing and bag connectors would thread together as usual, but in this implementation, an angled catch may slide past a foreshortened thread where it clicks into place, permanently locking the connectors together.

In other aspects, it may be preferable for patients to disconnect the drain bags from the drain line to make the bags easier to carry (e.g., individually carry) for disposal. In such aspects, another type of connector that alters the connection after the first time it is connected to system tubing may be preferred—this may include a one-time Luer-type lock or the like. This may include a mechanism inside the bag connector (e.g., the aforementioned ring with a notch) that moves in response to its connection with a system connector. For example, making the initial connection between the bag connector and fresh dialysate tubing may move a ring or other feature into a new position (see, e.g., FIGS. 8 and 9). Then, when disconnecting the bag from the tubing connection, the ring may be moved forward (e.g., unscrewed in a Luer type thread), so that it blocks a subsequent reconnection from being fully seated.

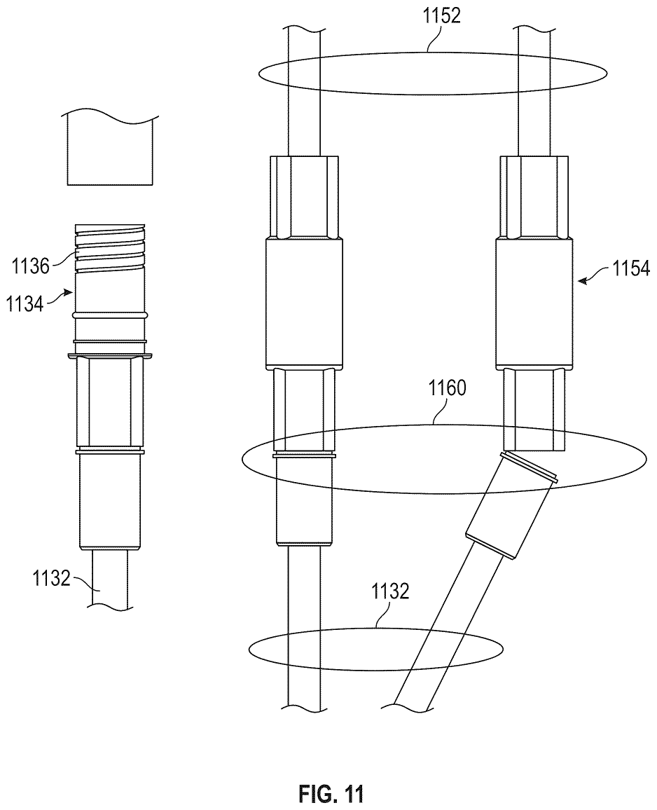

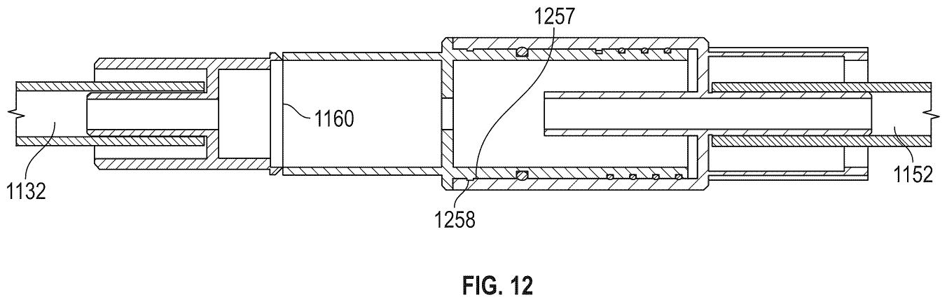

Another technique to prevent reuse of an effluent bag may include having one or more of the tubing connections be destructible so that they break off when trying to disconnect them from the initial connection to the solution/effluent bags—an example of such a mechanism is described below with reference to FIGS. 11 and 12.

FIG. 11 shows a first connector and system tubing, and FIG. 12 shows a first connector engaged with a system connector, according to representative examples. The connectors and tubing shown, and the system that these components can interact with, may be any as included herein, such as the system of FIGS. 1A-1D, the bag of FIG. 2, the connectors of FIGS. 5-10, or any of the techniques and methods described herein. The connectors shown in FIG. 11 may additionally include a breakpoint 1160. That is, this figure shows a first connector 1134 engaged with bag tubing 1132 that leads, directly or indirectly, to a bag, which again may be any as described herein. The first connector 1134 may include a first engagement portion 1136, which may be any as described herein. The first connector 1134 may include, or may be coupled (directly or indirectly) with a breakpoint 1160 that allows for separation of the bag and its bag tubing 1132 from another connector, i.e., a system connector 1154 as shown engaged with system tubing 1152.

The system connector 1154 may be any as described herein. In some aspects, the system connector 1154 is a supply side connector in a dialysis system. For example, in such aspects, the first connector 1134 and the system connector 1154 may permanently engage once fully seated relative to one another; after such engagement, the breakpoint 1160 may facilitate removal of the bag and its bag tubing 1132, e.g., for disposal of a substantially emptied or otherwise used bag. The remaining portion of the first connector 1134 still coupled to the system connector 1154 may prevent reuse of the bag as a source of dialysate in the system. In other aspects, the system connector 1154 is a drain side connector in a dialysis system. For example, in such aspects, the first connector 1134 and the system connector 1154 may permanently engage once fully seated relative to one another; after such engagement, the breakpoint 1160 may facilitate removal of the bag and its bag tubing 1132, e.g., for disposal after being used as a drainage receptacle. In such aspects, the disconnected breakpoint 1160 may prevent reuse of the bag as a source of dialysate in the system.

Thus, in aspects, one or more of the system connector 1154 (e.g., the supply side connector and/or a drain side connector) and the first connector 1134 include a breakpoint 1160 disposed away from one or more coupled portions of the first connector 1134 and the system connector 1154. The breakpoint 1160 may be thinner than at least one of the coupled portions of the first connector 1134 and the system connector 1154.

FIG. 12 shows a cutaway view of the connectors and respective tubing, i.e., the bag tubing 1132 and the system tubing 1152, for clarity. In particular, this figure shows an example of a first feature 1257 and a second feature 1258 as described herein—e.g., a second feature 1258 on a system connector (e.g., a supply side connector and/or a drain side connector) that is structurally configured to engage with a first feature 1257 on the first connector—e.g., a protrusion configured to engage with a notch, and/or a component designed to move a cooperating component. In some instances, the first connector includes a first feature 1257 and the system connector includes a second feature 1258, where the first feature 1257 and the second feature 1258 are configured to engage such that, when the first connector and the supply side connector are fully seated relative to one another, the first feature 1257 and the second feature 1258 permanently lock engagement therebetween. The second feature 1258 may be an angled barb or the like.

Thus, the present teachings may include a connector (e.g., a solution line connector, a drain line connector, a bag connector, etc.) that is formed (e.g., molded) with a thin point allowing the connector to break in two after a portion of a treatment is performed. The connector may feature an angled barb or the like along its chamber to provide for a permanent connection—i.e., once it is connected, it cannot be disconnected, preventing the reuse of bags with effluent. Once the bags are filled with effluent, a patient or other user may then break away the effluent bags from the tubing to make them easier to carry individually, where the broken ends cannot be reconnected to any tubing system.

The following example steps describe how an implementation with a destructible tubing connector may function. At the beginning of a treatment, a user may connect the tubing system to fresh solution bags using system connectors and bag connectors. At the end of a portion of the treatment where fresh dialysate is provided to a patient's peritoneum, the user may disconnect a downstream connector (e.g., by unsnapping an existing Vario connector or the like) to break away the tubing system connected to the empty solution bags. For the next portion of the treatment to drain the patient's peritoneum, the user may connect this “downstream” connector (e.g., the Vario connector) to a drain line connection of a new tubing set, i.e., to use the empty bags as drain bags. At the end of the draining portion of the treatment, the solution bags that were converted into drain bags may be full of effluent. If the user tries to separate the used solution bags/effluent bags from the tubing system, the breakpoint causes a portion of the connector to break off, leaving a portion permanently connected to the solution bag/effluent bag. This may prevent the solution bag/effluent bag from being reconnected to anything else—it is only fit for disposal now. Because the solution bag/effluent bag may be separable from the tubing system, the user may carry one bag at a time to a drain (e.g., toilet or tub) but may never reconnect the bag to any tubing by accident. In this manner, it may truly be a single reuse connector.

In certain implementations, the present teachings may include a bag for a peritoneal dialysis system. The bag may include: an exterior surface; a void in an interior of the bag structurally configured to be filled with a fluid; and a first connector in fluid communication with the void. The first connector may be structurally configured such that the bag is couplable only once to a supply side connector to transfer a supply of dialysis solution from the void to a peritoneum of a patient. The bag may further be structurally configured for fluidically coupling to a drain side connector after being coupled to the supply side connector such that the bag, when emptied, is a drainage receptacle for effluent but thereafter cannot subsequently be reconnected to supply side tubing.

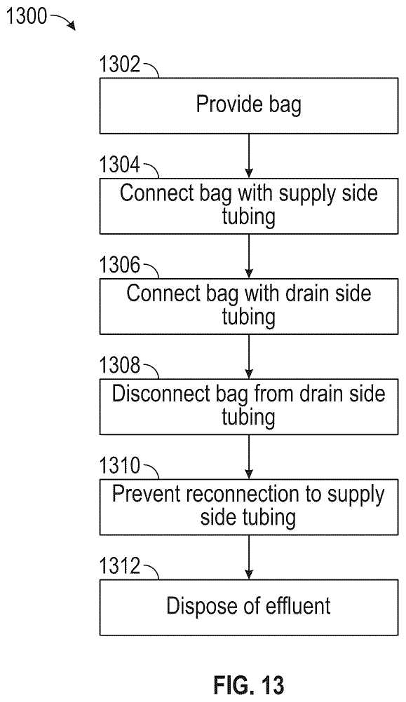

FIG. 13 is a flow chart of a method of preventing reuse of an effluent bag as a dialysate supply, according to a representative example. The method 1300 may be performed using any one or more of the devices and systems described herein.

As shown in step 1302, the method 1300 may include providing a bag structurally configured to connect with each of supply side tubing and drain side tubing in a peritoneal dialysis system. The bag may be any as described herein, and may include fresh dialysis solution.

As shown in step 1304, the method 1300 may include fluidically connecting the bag with the supply side tubing and supplying the fresh dialysis solution from the bag to a peritoneum of a patient during a first sequence of operation in a dialysis procedure, thereby substantially emptying the bag. Fluidically connecting the bag with the supply side tubing may include mechanically altering a first connector of the bag with a supply side connector such that, when the first connector is disengaged from the supply side connector, the first connector cannot be reengaged with the supply side connector. Fluidically connecting the bag with the supply side tubing may also or instead include permanently coupling a first connector of the bag with a supply side connector, where the peritoneal dialysis system further includes a tubing connector disposed downstream from the supply side connector, e.g., for connecting the bag with drain side tubing. Disconnecting the tubing connector from supply side tubing may also or instead include breaking at least a portion of the connection between the first connector and the supply side connector to separate the bag from tubing.

As shown in step 1306, the method 1300 may include fluidically connecting the bag with the drain side tubing and receiving effluent from the peritoneum of the patient during a second sequence of operation in the dialysis procedure, thereby substantially filling the bag with effluent. Fluidically connecting the bag with the drain side tubing may include disconnecting the tubing connector from supply side tubing and connecting the tubing connector to the drain side tubing, where disconnecting the bag from the drain side tubing includes decoupling the tubing connector from drain side tubing.

As shown in step 1308, the method 1300 may include disconnecting the bag from the drain side tubing. This may include breaking at least a portion of the connection between the first connector and a system connector to separate the bag from tubing. This may also or instead include disconnecting the first connector from the system connector, thereby mechanically altering at least one of the first connector and the system connector to prevent reengagement therebetween.

As shown in step 1310, the method 1300 may include preventing reconnection of the bag to the supply side tubing. This may be done by any one or more of the mechanical techniques described herein.

As shown in step 1312, the method 1300 may include disposing of effluent in the bag. To aid in this effort, the bags may be individually separated from their tubing connectors (the connector that was originally a supply side connector)—e.g., via a breakpoint or the like—such that carrying and/or emptying the bags is less of a burden.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings.

Unless the context clearly requires otherwise, throughout the description, the words “comprise,” “comprising,” “include,” “including,” and the like are to be construed in an inclusive sense as opposed to an exclusive or exhaustive sense; that is to say, in a sense of “including, but not limited to.” Additionally, the words “herein,” “hereunder,” “above,” “below,” and words of similar import refer to this application as a whole and not to any particular portions of this application.

It will be appreciated that the devices, systems, and methods described above are set forth by way of example and not of limitation. Absent an explicit indication to the contrary, the disclosed steps may be modified, supplemented, omitted, and/or re-ordered without departing from the scope of this disclosure. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the implementations described herein are intended to include any suitable method of causing such method steps to be performed, consistent with the patentability of the following claims, unless a different meaning is expressly provided or otherwise clear from the context. So, for example performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A peritoneal dialysis system, the system comprising:
supply side tubing with a supply side connector structurally configured to fluidically connect to a supply of dialysis solution to transfer the dialysis solution to a peritoneum of a patient;
drain side tubing with a drain side connector structurally configured to fluidically connect to a drainage receptacle to transfer effluent from the peritoneum of the patient to the drainage receptacle; and
a bag comprising a first connector structurally configured such that the bag is couplable only once to the supply side connector such that the bag, when filled with fresh dialysis solution, is configured to be the supply of dialysis solution in the system, the bag further structurally configured for fluidically coupling to the drain side connector after being coupled to the supply side connector such that the bag, when emptied, is configured to be the drainage receptacle for effluent but thereafter cannot subsequently be reconnected to the supply side tubing.

2. The system of claim 1, wherein the first connector is mechanically altered upon engagement with the supply side connector such that, upon disengagement from the supply side connector, the first connector cannot be reengaged with the supply side connector.

3. The system of claim 1, wherein the first connector includes a first feature and the supply side connector includes a second feature, the second feature structurally configured to engage with the first feature and to move the first feature from an initial position to a final position through one or more of an engagement operation and a disengagement operation, wherein the final position of the first feature prevents reengagement between the first connector and the supply side connector.

4. The system of claim 3, wherein the first feature includes a ring with a notch, and wherein the second feature includes a tab, wherein the tab is engageable with the notch when the first connector and the supply side connector are fully seated relative to one another, and wherein, when the supply side connector is disengaged from the first connector, the tab moves the ring outward towards an opening in the first connector thereby at least partially blocking the opening from receiving the supply side connector.

5. The system of claim 3, wherein the first feature is configured to move along a length of the first connector in a direction substantially parallel to a central axis of the first connector from a first axial position to a second axial position, the first axial position more proximal to a base portion of the first connector, and more distal to an opening of the first connector, than the second axial position.

6. The system of claim 5, wherein the first feature is prevented from moving back to the first axial position once positioned in the second axial position.

7. The system of claim 3, wherein the first feature comprises one or more of a ring-like structure, a collar, a sleeve, an annular member, a segmented ring structure, a C-shaped element, and multiple discrete elements arranged circumferentially within the first connector.

8. The system of claim 1, wherein the first connector, when engaged with the supply side connector, becomes permanently coupled to the supply side connector, and wherein the system further comprises a downstream connector that is couplable to the drain side connector for use of the bag, when emptied, as the drainage receptacle.

9. The system of claim 8, wherein the first connector includes a first feature and the supply side connector includes a second feature, the first feature and the second feature configured to engage such that, when the first connector and the supply side connector are fully seated relative to one another, the first feature and the second feature permanently lock engagement therebetween.

10. The system of claim 9, wherein the first feature includes a notch and the second feature includes a catch.

11. The system of claim 9, wherein one or more of the supply side connector and the first connector include a breakpoint disposed away from one or more coupled portions of the first connector and the supply side connector.

12. The system of claim 11, wherein the breakpoint is thinner than at least one of the one or more coupled portions of the first connector and the supply side connector.

13. The system of claim 1, further comprising a chemical sensor in an interior of the bag, the chemical sensor reactive with one or more contents of the effluent but non-reactive with the fresh dialysis solution.

14. The system of claim 13, wherein the chemical sensor includes, or is in communication with, a visual indicator that changes from a first visual state to a second visual state when the chemical sensor reacts with the one or more contents of the effluent to visually indicate that the bag contains waste.

15. A method of preventing reuse of an effluent bag as a dialysate supply, the method comprising:
providing a bag structurally configured to connect with each of supply side tubing and drain side tubing in a peritoneal dialysis system, the bag comprising fresh dialysis solution;
fluidically connecting the bag with the supply side tubing and supplying the fresh dialysis solution from the bag to a peritoneum of a patient during a first sequence of operation in a dialysis procedure, thereby substantially emptying the bag;
fluidically connecting the bag with the drain side tubing and receiving effluent from the peritoneum of the patient during a second sequence of operation in the dialysis procedure, thereby substantially filling the bag with effluent;
disconnecting the bag from the drain side tubing; and
preventing reconnection of the bag to the supply side tubing.

16. The method of claim 15, wherein fluidically connecting the bag with the supply side tubing includes mechanically altering a first connector of the bag with a supply side connector such that, when the first connector is disengaged from the supply side connector, the first connector cannot be reengaged with the supply side connector.

17. The method of claim 15, wherein fluidically connecting the bag with the supply side tubing includes permanently coupling a first connector of the bag with a supply side connector, the peritoneal dialysis system further including a tubing connector disposed downstream from the supply side connector.

18. The method of claim 17, wherein fluidically connecting the bag with the drain side tubing includes disconnecting the tubing connector from supply side tubing and connecting the tubing connector to the drain side tubing, and wherein disconnecting the bag from the drain side tubing includes decoupling the tubing connector from drain side tubing.

19. The method of claim 17, further comprising breaking at least a portion of the connection between the first connector and the supply side connector to separate the bag from tubing.

20. A bag for a peritoneal dialysis system, the bag comprising:
an exterior surface;
a void in an interior of the bag structurally configured to be filled with a fluid; and
a first connector in fluid communication with the void, the first connector structurally configured such that the bag is couplable only once to a supply side connector to transfer a supply of dialysis solution from the void to a peritoneum of a patient, wherein the bag is structurally configured for fluidically coupling to a drain side connector after being coupled to the supply side connector such that the bag, when emptied, is a drainage receptacle for effluent but thereafter cannot subsequently be reconnected to supply side tubing.
